(19) United States
(12) Patent Application Publication
(10) Pub. No.: US 2022/0362354 A1
(43) Pub. Date: Nov. 17, 2022

(54) WIDE-SPECTRUM ANTIBACTERIAL PHARMACEUTICAL FORMULATIONS COMPRISING LYSOZYME AND METHODS OF USING THE SAME

(71) Applicant: AYBAR ECOTECHNOLOGIES CORP., Santo Domingo (DO)

(72) Inventor: Diogenes AYBAR-BATISTA, Santo Domingo (DO)

(73) Assignee: AYBAR ECOTECHNOLOGIES CORP., Santo Domingo (DO)

(21) Appl. No.: 17/875,872

(22) Filed: Jul. 28, 2022

Related U.S. Application Data
(63) Continuation of application No. 16/885,537, filed on May 28, 2020, now abandoned.
(60) Provisional application No. 62/853,215, filed on May 28, 2019.

(51) Int. Cl. A61K 38/47; A61K 47/18; A61K 47/02

(52) U.S. Cl. CPC A61K 38/47; A61K 47/183; A61K 47/02; A61K 9/0019

(57) ABSTRACT

A wide-spectrum antibacterial pharmaceutical formulation comprising lysozyme and methods of treatment to prevent or cure diseases of bacterial etiology. Excipients provided in the pharmaceutical formulation comprising lysozyme enhance the stability and efficacy of lysozyme for treating bacterial infections in a mammal. The pharmaceutical formulation comprising lysozyme does not produce harmful secondary effects on tissues or organs during prolonged treatment. The formulation can be used to treat bacterial infections of the skin, mucosal regions, and administered into the blood stream of a patient, including respiratory infections. The formulation is useful in treating bacterial infections, including those that occur along with viral infections, particularly viral infections with a respiratory component, including COVID-19.

### CROSS-REFERENCE TO RELATED APPLICATION

This application is a continuation of U.S. patent application Ser. No. 16/885,537, filed on May 28, 2020, and claims priority to U.S. Provisional Patent Application Ser. No. 62/853,215, filed on May 28, 2019, which are hereby incorporated by reference in their entirety into the present application.

### FIELD OF INVENTION

The present invention relates generally to a wide-spectrum bactericidal formulation for the treatment or prevention of bacterial infections in a mammal, including bacterial infections that accompany viral infections. The invention also includes methods of treating bacterial infections in a mammal by administering to an infected area of the mammal a pharmaceutical composition of the present invention.

### DISCUSSION OF THE BACKGROUND

Presented below is background information on certain aspects of the present invention as they may relate to technical features referred to in the detailed description, but not necessarily described in detail. That is, certain components of the present invention may be described in greater detail in the materials discussed below. The discussion below should not be construed as an admission as to the relevance of the information to the claimed invention or the prior art effect of the material described.

Bacterial resistance to antibiotic small molecule drugs is a growing medical problem that will eventually reduce or eliminate many treatment options for bacterial infections, leaving patients susceptible to previously treatable conditions. Antibacterial small molecule drugs typically work by targeting a particular bacterial enzyme to block a critical biosynthetic pathway necessary to allow bacteria to multiply or survive internal and external stresses. Bacteria often develop genetic resistance to these drugs by modifying the enzymatic target sites of small molecule drugs. This resistance is in turn passed onto bacterial progeny very quickly creating new populations of antibiotic resistant strains and substrains. The World-Health Organization estimates that even as new antibiotic drugs are developed global antibiotic resistance will remain a major threat.

Additionally, antibacterial formulations are known to produce unintended negative side-effects impairing the health of the patient being treated (Cunha, Burke A. “Antibiotic side effects.” Medical Clinics of North America 85.1 (2001): 149-185). Most side-effects associated with antibiotic treatments are not life-threatening. However, these side-effects can reduce patient compliance for completing the prescribed treatment courses, thereby contributing to bacterial resistance in the global population. For example, commonly prescribed drugs, such as tetracyclines often induce photo-sensitivity in patients, whereas patients taking beta-lactams often suffer fevers or in some cases potentially life-threatening allergic reactions.

With respect to viral and bacterial respiratory co-infections, the elimination of the bacterial population (both, on the onset of the viral infection, and during the advanced stages of the viral infection) is crucial for avoiding or diminishing critical respiratory conditions and mortality.

Accordingly, there is a critical and unmet need for a wide-spectrum bactericide formulation that can be used to cure or prevent a wide range of diseases of bacterial etiology without the problems often associated with antibacterial drugs.

### SUMMARY OF THE INVENTION

The following brief summary is not intended to include all features and aspects of the present invention, nor does it imply that the invention must include all features and aspects discussed in this summary.

The present disclosure overcomes previous problems associated with antibiotic drugs by using a universal bactericide formulation comprising lysozyme and one or more divalent metal chelating agents as a cofactor to enhance the efficacy of lysozyme for treating bacterial infections in a mammal. The formulation provided herein uses lysozyme and excipients that are safe and minimize unintended negative side-effects. The present disclosure further includes methods of treating various infections of bacterial etiology in a mammal using lysozyme formulations.

Lysozyme is the most prominent member of the very large class of glycosidases or glycohydrolases, enzymes that catalyze the transfer of a glycosyl group to water. Lysozyme catalyzes the hydrolysis of a polysaccharide component of the cell wall of Gram-positive bacteria. To do this it accelerates the cleavage of a glycosidic C—O bond between N-acetylmuramic acid and N-acetyl-D-glucosamine residues in the peptidoglycan component of the cell wall. The early crystal structure work of lysozyme showed that the enzyme binds the substrate in such a way that the atoms of the target C—O bond come within reach of two, and only two, potential catalytic groups, Glu 35 and Asp 52.

Lysozyme carries out its antibacterial activity by contacting bacteria and disrupting the cell wall formed over the phospholipid membrane. The bacterial cell wall protects them against osmotic pressure between the inside and the outside of the cell that can induce detrimental cellular stresses including lysis. Bacteria can be classified, according to the architecture of their cell walls, into Gram negative and Gram positive. Lysozyme is known to be a bactericide for most of the Gram positive bacteria, but weakly active against Gram negative strains. This is primarily due to the fact that Gram negative bacteria include a lipopolysaccharide (LPS) containing outer membrane covering the peptidoglycan layer found between the outer and inner membrane.

In the presence of divalent cation chelating agent, like EDTA, lysozyme becomes as effective against Gram negative as against Gram positive bacteria. The mechanism for this effect of chelating agents is not well understood. However, it is hypothesized that removal of stabilizing divalent cations from the LPS layer by chelating agents results in the release of LPS, allowing molecules to penetrate the outer membrane.

Lysozyme and EDTA are Generally Recommended as Safe (GRAS) by the FDA. The present inventors have found that lysozyme as an active component in a therapeutic formulation provides several advantages including, but not limited to, being easy to produce and administer to patients, while also being highly safe and tolerable to patients through various routes of administration.

Lysozyme is active in a wide range of pH values, but its optimal pH is range is between pH 4 and pH 6. In fact, lysozyme shows stable catalytic efficiency over this entire pH range.

In some embodiments of the present disclosure lysozyme acts as the main bactericidal component in the bactericidal formulation by disrupting the cell wall of the bacteria infecting the mucous membrane or surrounding environment of the region infected. In some embodiments, the bactericidal formulation will include a chelating agent that will enhance the effect of lysozyme against Gram positive and Gram negative bacteria. In some embodiments the bactericidal formulation will include a pH stabilizing agent to ensure: 1) that the pH of the product, once dissolved in the region of action, is not harmful to the patient; and 2) that, once the product is dissolved in the region of action, the pH of the solution produced remains always within the range optimum for lysozyme activity. In still other embodiments, a filling of natural and neutral substance may be added for diluting the active ingredients of the formula to the proper concentration for its topical action. In an embodiment, natural flavors or colorants are added to the formulation.

In some embodiments the bactericidal formulation is a pharmaceutical powder formulation for administration to a mammal comprising lysozyme, a pharmaceutically acceptable chelating agent, and a pH stabilizing salt. The pharmaceutical powder formulation of the invention may be dissolved in an aqueous solution prior to administration to a mammal. In some embodiments the pharmaceutical powder formulation will include zinc oxide. In still other embodiments the pharmaceutical powder formulation will include magnesium citrate.

In certain embodiments the bactericidal formulation is in the form of a tablet for oral administration comprising lysozyme, a pharmaceutically acceptable chelating agent, a pH stabilizing salt, and a thickener and/or excipient comprising a resin. In an embodiment the pharmaceutical tablet formulation includes at least one of a flavoring agent, a coloring agent, or a combination thereof. In still other aspects, the pharmaceutical tablet formulation is in the form of a chewable tablet for oral administration.

In certain aspects of the invention the pH stabilizing salt has a buffering capacity in the range required to maintain optimal catalytic efficiency of lysozyme. In preferred embodiments, the pH stabilizing salt has a buffering capacity within the range of pH 3.0 to about pH 7.0. More preferably the pH stabilizing salt stabilizes the pH of the formulation in solution at a pH of about pH 6.0 to about pH 6.8.

The inventors have found that specific agents may be included in the formulation of the present disclosure that act synergistically with the lytic activity of lysozyme. Accordingly, in some embodiments of the present disclosure the bactericidal formulation comprising lysozyme will include a synergic component. In some embodiments, the synergic component is solubilizing agent. In an embodiment the formulation is in a solid (powder or tablet) state before administering, for example, for oral use, and a mixture of citric acid and sodium bicarbonate can be used to accelerate its dissolution. In some embodiments, the synergic component is a drying agent or antiseptic carrier. In an embodiment of the formulation for external (skin) applications, a carrier may be utilized with a fourfold function: a) carrier, b) keeping the area of application dry, c) antiseptic agent to maintain the condition of the exterior portion of the treated area, and d) releasing the bactericide components of the formulation into the interior of the treated area when entering in contact with any wet region.

In some aspects of the invention, the pharmaceutical formulation of the present invention may be used to treat bacterial infections of the skin. For example, the pharmaceutical formulation may be used to treat or prevent infections in skin sores persistent in diabetic and immunologically debilitated patients; skin burns (at different levels) which often become infected because of the exposure to the environment and cause the patient to undergo a painful recovery procedure and other bacterial skin infections.

In an aspect of the invention, the pharmaceutical formulation disclosed herein may be used to treat bacterial infections of the oropharyngeal, pylorus and esophageal tissue areas. Permanent incubation regions for bacteria lie in the sinuses, oral cavity (e.g., the gums) and in the throat, causing persistent infection in the mucosal tissue surrounding these regions. Since these areas have a common port of entry, the pharmaceutical formulation of the present invention allows cotemporaneous treatment of these areas.

In some embodiments the pharmaceutical formulation may be used to treat bacterial infections of the large intestine, including acute Salmonellosis, Colitis and Diverticulitis. In some embodiments the main component of the formulation is lysozyme present in sufficient quantity, and administered in a high volume carrier (such as water), that a pharmaceutically effective amount of enzyme is provided the large intestine. In non-limiting embodiments the formulation includes an agent that induces intestinal flushing promoting the flow of water into the large intestine forcing the bacteria into the liquid suspension that forms immediately in the intestine cavity. This allows lysozyme to easily attack and destroy the bacteria (a purgative effect).

In certain embodiments the pharmaceutical formulation may be used to treat bacterial infections present in the upper and lower respiratory tissues, including the sinuses and lungs. These two regions have a common port of entry, but at the same time often sinuses become incubation zones that allow for reinfection. In another aspect of the invention the pharmaceutical formulation provided herein may be used to treat or cure bacterial infections in the blood stream or vital organs of a mammal. Sinus, lungs, blood and the vital inner organs are very delicate and sensitive to any non-soluble materials. Thus, some aspects of the present invention provide formulations containing only lysozyme and ethylenediaminetetraacetic acid (EDTA). In some embodiments sodium bicarbonate is provided with EDTA in acid form. In yet other embodiments, the pharmaceutical formulation includes EDTA salt and minute amounts of sodium bicarbonate to stabilize the pH of the formulation after it is dissolved in an aqueous solution.

In further aspects, the formulation of the present invention may be used during surgical procedures, as an irrigant or cleansing solution produce asepsis in the area of surgical intervention, or during the post-surgery recovery period. As a result, the formulation disclosed herein may eliminate the need for using antibiotics during surgery or post-surgical recovery periods. It will be readily apparent to those skilled in the art that because of the bactericidal action of the formulation comprising lysozyme during surgery and the adequate use during the post-surgery recovery period, the need for using anti-inflammatory drugs during the post-surgery recovery period may be eliminated.

In still other aspects of the present invention, the pharmaceutical formulation may be used to treat a bacterial infection of the eye, for example, bacterial conjunctivitis.

The foregoing and other objects, features and advantages of the invention will be apparent from the following more particular description of preferred embodiments of the invention, as illustrated in the accompanying drawings. The drawings illustrate the principles of the invention, but the invention of the present disclosure is not limited thereto.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph showing the Silness and Loe gingival index for patients treated with Lysodent™ versus Chlorhexidine.

FIGS. 2A-B are graphs showing gingivitis-related inflammation in patients treated with Lysodent™ for days 1-28 in FIG. 2A and gingivitis-related bleeding in patients treated with Lysodent™ for days 1-28 in FIG. 2B.

FIGS. 3A-B are graphs showing gingivitis-related inflammation in patients treated with Chlorhexidine for days 1-28 in FIG. 3A and gingivitis-related bleeding in patients treated with Chlorhexidine for days 1-28 in FIG. 3B.

FIGS. 4A-B are graphs showing gingivitis-related inflammation observed in patients treated with Chlorhexidine in FIG. 4A or Lysodent™ in FIG. 4B.

FIGS. 5A-B are graphs showing gingivitis-related bleeding observed in patients treated with Chlorhexidine in FIG. 5A or Lysodent™ in FIG. 5B.

FIG. 6 is a graph showing pain reported in patients treated with Chlorhexidine or Lysodent™ following dental extraction surgery.

FIG. 7. is a graph showing inflammation reported in patients treated with Chlorhexidine or Lysodent™ following dental extraction surgery.

FIG. 8. is a graph showing patient reaction to formulation flavor with Chlorhexidine or Lysodent™ following dental extraction surgery.

FIG. 9. is a graph showing patient flavor perception changes following treatment with Chlorhexidine or Lysodent™ following dental extraction surgery.

FIG. 10. is a graph showing pain reported in patients treated with Chlorhexidine or Lysodent™ following dental implant surgery.

FIG. 11. is a graph showing inflammation reported in patients treated with Chlorhexidine or Lysodent™ following dental implant surgery.

FIG. 12. is a graph showing bleeding reported in patients treated with Chlorhexidine or Lysodent™ following dental implant surgery.

FIG. 13. is a graph showing patient reaction to formulation flavor with Chlorhexidine or Lysodent™ following dental implant surgery.

FIG. 14. is a graph showing patient flavor perception changes following treatment with Chlorhexidine or Lysodent™ following dental implant surgery.

FIG. 15. is a schematic view of the teeth evaluated for the Silness and Loe gingival index.

### DETAILED DESCRIPTION OF PREFERRED EMBODIMENTS

The following detailed description includes references to the accompanying drawings, which form part of the detailed description. The drawings show, by way of illustration, a specific embodiment by which the present invention may be practiced. The embodiments herein may be combined, other embodiments may be utilized, or changes may be made based on structural, chemical, or other logical changes that are within the scope of the present invention. Therefore, the following detailed description is not to be taken as limiting in scope.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition terms not specifically used. For example, amending a term from “comprising” to “consisting essentially of” would find direct support due to this definition.

The term “about” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

Generally, herein, the term “or” includes “and/or.”

As used herein, a plurality of compounds or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “At least one” is not to be construed as limiting “a” or “an.” “Or” means “and/or.” As used herein, the term “and/or” includes any and all combinations of one or more of the associated listed items.

A “pharmaceutically acceptable chelating agent” means an agent that is safe and effective for administering to a mammal, including a human, and produces minimal or no negative side-effects, alone or in combination with other pharmaceutical components.

A “pharmaceutically acceptable aqueous solution” includes aqueous solutions that are suitable for administration to mammals, e.g., humans, including, but not limited to, topical, oral, subcutaneous, or intravenous administration.

The phrase “pharmaceutically acceptable amount” of the present invention refers to an amount of either lysozyme as the active agent or the total amount of the pharmaceutical composition comprising lysozyme that will treat or cure a bacterial infection in the affected area.

“Buffering capacity” of a pH stabilizing salt is the ability of the salt to resist pH change in response to the addition of a strong acid or base in solution. For example, buffering capacity may be measured according to the amount of strong acid or base required to change the pH of one liter of the solution by one pH unit under standard conditions of temperature and pressure. Empirical methods for determination of the buffering capacity of a given salt by both acid titration and base titration over a given range of pH change from the desired pH of the composition include conventional techniques that are well-known to those skilled in the art.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The inventors of the present disclosure have demonstrated surprisingly that a pharmaceutical formulation comprising lysozyme as provided herein acts as a universal bactericide, killing both Gram negative and Gram positive bacteria. The pharmaceutical formulation of the present disclosure does not produce harmful side effects on human tissue and organs, even during prolonged use.

As a non-limiting example, the inventors of the present invention have found that a suitable pharmaceutical formulation according to the present invention is a 50 mg powder formulation. In some embodiments the amount of lysozyme is in the range of about 1 mg to about 45 mg. In some aspects of the invention the amount of lysozyme is about 10 mg or more, or about 20 mg to about 40 mg, or optimally about 35.6 mg of lysozyme. In some aspects of the invention, the formulation comprises about 1 mg to about 45 mg of a chelating agent. In other aspects the amount of chelating agent is about 5 mg to about 20 mg, or about 10 to about 20 mg, or optimally about 12.2 mg of chelating agent. In some embodiments the amount of pH stabilizing salt is about 0.1 mg to about 10 mg. In some aspects of the invention the amount of pH stabilizing salt is about 0.5 mg to about 5.0 mg, or about 1.0 mg to about 2.0 mg, or optimally about 1.3 mg of a pH stabilizing salt. In some embodiments, 100 mg of a powder formulation according to the present invention (twice the forgoing amounts, respectively) is dissolved in 10 ml of aqueous solution. In other embodiments, 250-300 mg of a powder formulation according to the present invention (five times and six times the foregoing amounts, respectively) is dissolved in 1.0 liter of an aqueous solution. In yet other aspects of the present invention, 300, 400, 500, 700 or 1000 mg of a powder formulation, as described above, may be used per liter of aqueous solution.

In some embodiments, the 50 mg powder formulation described above includes zinc oxide at a weight ratio of about 1:99 to about 50:50 total lysozyme formulation (for example, 50 mg of the lysozyme powder formulation) to zinc oxide. In other aspects, the ratio of lysozyme formulation to zinc oxide is about 5:95 to about 20:80, and in some aspects of the invention the amount of lysozyme formulation to zinc oxide is about 10:90 to about 30:70.

In a further non-limiting example, the inventors have found that a suitable pharmaceutical formulation according to the present invention is a 500 mg tablet formulation. In some embodiments the amount of lysozyme is in the range of about 1 mg to about 100 mg. In some aspects of the invention the amount of lysozyme is about 10 mg to about 50 mg, or about 30 mg to about 40 mg, or optimally about 35.6 mg of lysozyme. In some aspects of the invention, the formulation comprises about 1 mg to about 50 mg of a chelating agent. In other aspects the amount of chelating agent is about 5 mg to about 40 mg, or about 10 mg to about 20 mg, or optimally about 12.2 mg of chelating agent. In still other aspects of the invention, a pH stabilizing salt is added in the amount of about 1 mg to about 10 mg. In certain aspects, the amount of the pH stabilizing salt is about 2.5 mg to about 5 mg, or about 3.5 mg to about 4.5 mg, or optimally about 4.0 mg of a pH stabilizing salt. In addition, some embodiments of the formulation comprises about 10 mg to about 450 mg of a pharmaceutically acceptable resin. In other aspects, the formulation includes about 100 mg to about 400 mg, or about 200 mg to about 390 mg, or about 446.6 mg of a pharmaceutically acceptable resin. In some embodiments, the formulation includes about 0.1 mg to about 10 mg of a flavoring agent and/or, separately, a coloring agent in an equal or different amount. Optimally, the formulation can include about 0.3 mg or about 0.5 mg of a flavoring agent and/or coloring agent in an equal or different amount. In an aspect of the invention the tablet is a chewable tablet.

In a preferred embodiment, the pharmaceutical formulation is a powder comprising lysozyme and a chelating agent. The inventors of the present disclosure have found that lysozyme is safe and effective for treating a wide range of bacterial infections when lysozyme is present in relatively low concentrations in solution. In some embodiments, the amount of lysozyme is present in an amount of about 0.2% by weight to about 90% by weight, or about 2% by weight to about 80% by weight, or about 6% by weight to about 60% by weight, or about 8% by weight to about 40% by weight, or about 10% by weight to about 20% by weight, or about 12% by weight to about 15% by weight based on the total weight of the pharmaceutical formulation. In an aspect of the invention, lysozyme is present in an amount of about 73% by weight or less based on the total weight of the pharmaceutical formulation. The present disclosure is not limited by the specific amount of lysozyme listed herein and may use any amount of lysozyme between the foregoing ranges.

Pharmaceutically acceptable chelating agents may be selected from ethylene diamine tetra-acetic acid (EDTA) salt, citrate salt, alginate salt, and a combination thereof. The inventors have found that an amount of chelating agent useful for providing the broad spectrum antibacterial activity of the pharmaceutical formulation of the present disclosure may be present in a wide range of concentrations. In some embodiments the amount of chelating agent is in an amount of about 0.2% by weight to about 90% by weight, about 2% by weight to about 40% by weight, about 4% by weight to about 24% by weight, or about 10% by weight to about 20%, or about 12% by weight to about 15% by weight based on the total weight of the pharmaceutical formulation.

In an embodiment of the present invention, the pharmaceutical formulation further includes a pharmaceutically acceptable pH stabilizing salt. The pH stabilizing salt is selected from the group consisting of citrate salt and sodium bicarbonate. The inventors have found that an amount of pH stabilizing salt useful for maintaining a safe and effective pH when the pharmaceutical formulation is dissolved in an aqueous solution is about 0.2% by weight to about 20% by weight. In some aspects, the amount of pH stabilizing salt can be present in an amount of about 0.5% by weight to about 4% by weight, about 0.7% by weight to about 2.6% by weight, about 0.8% by weight to about 2% by weight, or about 0.9% by weight to about 1.0% by weight based on the total weight of the pharmaceutical formulation.

In one aspect of the present invention, the pharmaceutical formulation will comprise a resin. Non-limiting examples of a resin useful in the formulation of the present disclosure include vegetable-based resins. A preferred resin for the pharmaceutical formulation is an alginic acid resin. In some embodiments resin is present in an amount of 2% by weight to about 90% by weight. In other embodiments the resin is present in an amount of about 20% by weight to about 89% by weight, about 40% by weight to about 80%, or about 50% by weight to about 70% by weight based on the total weight of the pharmaceutical formulation. In some aspects of the invention the resin is present in an amount of about 78% present by weight based on the total weight of the pharmaceutical formulation.

In some aspects of the invention the formulation further includes magnesium citrate. In certain embodiments magnesium citrate is included in an amount of about 0.1 grams to about 5.0 grams. In other aspects of the invention, magnesium citrate is included in the formulation in an amount of about 0.5 grams to about 2.5 grams, or about 1.0 gram to about 2.0 grams.

In another aspect of the invention, the pharmaceutical formulation includes a coloring agent, a flavoring agent, or a combination thereof. The coloring agent and/or flavoring agent is included in an amount of about 0.02% by weight to about 2% by weight, equally or differently based on the total weight of the pharmaceutical formulation. In other aspects of the invention, the coloring agent and/or flavoring agent is present in an amount of about 0.06% by weight to about 1% by weight, equally or differently based on the total weight of the pharmaceutical formulation.

The pharmaceutical formulation of the present disclosure can be dissolved in an aqueous solution prior to administration to a mammal. In some embodiments, the pharmaceutical formulation is dissolved in water, or sterile and/or deionized water. In other embodiments, the pharmaceutical formulation is dissolved in a physiologic saline solution.

The present disclosure provides a method of treating a bacterial infection in the digestive tract in a mammal, comprising orally administering (i.e., ingesting) a pharmaceutical powder formulation or effervescent tablet dissolved in water, wherein said powder formulation comprises: (i) lysozyme, (ii) magnesium citrate; (iii) a pharmaceutically acceptable chelating agent, (iv) a pharmaceutically acceptable pH stabilizing salt, wherein the pH stabilizing salt has buffering capacity in the range of pH 3.0 to pH 7.0. In non-limiting embodiments, the formulation is dissolved in about 500 mL to about 700 mL of water. In some embodiments citric acid and sodium bicarbonate are included in a stoichiometric amount. Preferably, the method includes administering the entire solution such that the patient ingests the total volume at once. In some embodiments of this method, a second dose is administered 8 hours after administration of the first dose.

The present disclosure provides a method of treating a bacterial infection of the oral cavity in a mammal, comprising orally administering a chewable pharmaceutical tablet, wherein said tablet comprises: (i) lysozyme, (ii) a pharmaceutically acceptable resin; (iii) a pharmaceutically acceptable chelating agent, (iv) a pharmaceutically acceptable pH stabilizing salt, the pH stabilizing salt has buffering capacity in the range of pH 3.0 to pH 7.0, and optionally a coloring agent, a flavoring agent, or a combination thereof. The resin thickening agent is added to achieve a viscous mixture in the mouth when the tablet is chewed such that, when the solution is swallowed, the solution moves slowly down the throat and the esophagus, thus augmenting the resident time in those regions rendering the formulation more effective in its action of killing the bacteria that it encounters in its course down to the stomach. In some embodiments a stoichiometric mixture of citric acid and sodium bicarbonate is added in an amount of about 5% by weight based on the total weight of the formulation in order to enhance the dissolution of the solid tablet when in contact with the patient's saliva. In some aspects of this method, the formulation is administered once every 12 hours.

The present disclosure further provides a method of treating a bacterial respiratory tract infection in a mammal, comprising introducing a pharmaceutical formulation into the respiratory tract of a mammal, wherein said pharmaceutical formulation comprises an aqueous solution comprising: (i) lysozyme, (ii) a pharmaceutically acceptable chelating agent, (iii) a pharmaceutically acceptable pH stabilizing salt, wherein the pH stabilizing salt has buffering capacity in the range of pH 3.0 to pH 7.0, and wherein the solution is administered using a nebulizer. In some embodiments 50-100 mg of the pharmaceutical formulation is dissolved in 5.0 mL of an aqueous solution comprising physiological saline or distilled water. In some embodiments, the nebulizer is used to treat the lungs of an infected mammal every eight hours until the composition has been applied three times.

Another aspect of the invention is a method of treating septicemia in a mammal, comprising introducing a pharmaceutical formulation into the blood stream of a mammal, wherein said pharmaceutical formulation comprises a physiologic saline solution comprising: (i) lysozyme, (ii) a pharmaceutically acceptable chelating agent, (iii) a pharmaceutically acceptable pH stabilizing salt, wherein the pH stabilizing salt has buffering capacity in the range of pH 3.0 to pH 7.0, and wherein the solution is administered intravenously. In some embodiments, the saline solution comprises about 100 mg to about 1000 mg of the pharmaceutical composition per liter of solution.

The present disclosures additionally provide a method of treating a bacterial skin infection in a mammal, comprising contacting the affected area of the skin with a physiological saline solution, comprising (i) lysozyme, (ii) a pharmaceutically acceptable chelating agent, (iii) a pharmaceutically acceptable pH stabilizing salt, wherein the pH stabilizing salt has buffering capacity in the range of pH 3.0 to pH 7.0. In some aspects of this invention, this method further comprises drying the affected area of the skin; and applying to the affected area of the skin a pharmaceutical powder formulation comprising: (i) lysozyme; (ii) a pharmaceutically acceptable chelating agent; (iii) a pharmaceutically acceptable pH stabilizing salt, (iv) and zinc oxide; wherein the pH stabilizing salt has buffering capacity in the range of pH 3.0 to pH 7.0.

Provided herein is a method of treating or preventing bacterial infections in a mammal undergoing a surgical procedure, comprising contacting an area of surgical intervention with a physiological saline solution, comprising (i) lysozyme, (ii) a pharmaceutically acceptable chelating agent, (iii) a pharmaceutically acceptable pH stabilizing salt, wherein the pH stabilizing salt has buffering capacity in the range of pH 3.0 to pH 7.0. In some embodiments, the method further comprises applying a physiological saline solution of this method to the suture site after the surgical procedure. In some embodiments, the method further comprises applying to the affected area of the skin after the surgical procedure a pharmaceutical powder formulation comprising: (i) lysozyme; (ii) a pharmaceutically acceptable chelating agent; (iii) a pharmaceutically acceptable pH stabilizing salt, (iv) and zinc oxide; wherein the pH stabilizing salt has buffering capacity in the range of pH 3.0 to pH 7.0.

Examples of bacterial infections that may be treated using the compounds of the invention include, but are not limited to Atopobium (e.g., parvulum, rimae), Bacillus (e.g., anthracis), Bacteroides (e.g., fragilis), Bordetella (e.g., pertussis), Borrelia burgdorferi, Bulleidia extructa, Campylobacter (e.g., jejuni), Catonella morbi, Centipeda periodontii, Chlamydia (e.g., trachomatis), Clostridium (e.g., difficile, hastiforme, histolyticum, perfringens, subterminale, clostridioform, sporogenes, bifermentans, botulinum, oedematiens, welchii, tetani), Cryptobacterium curtum, Dialister pneumosintes, Escherichia coli, Eubacterium sulci, Filifactor alocis, Fusobacterium (e.g., periodonticum, nucleatum), Granulicatella adiacens, Haemophilus influenzae, lactobacillus, Listeria (e.g., monocytogenes), Mogibacterium (e.g., timidum, vescum), Mycobacterium (e.g., tuberculosis), Neisseria (e.g., gonorrhoeae), Prevotella, Porphyromonas (endodontalis, gingivalis), Pneumococcus, Pseudoramibacter alactolyticus, Salmonella, Selenomonas sputigena, Shigella, Slackia exigua, Staphylococcus (e.g., aureus [MRSA], epidermidis), Streptococcus (e.g., pneumoniae, mitis, oralis, salivairus, sanguinis, milleri, mutans, sobrinus, anginosus), Tannerella forsythia, Treponema (e.g., denticola, socranskii, pallidum, pectinovorum, amylovorum, medium), Vibrio (e.g., cholerae).

Also provided herein is a method of treating or preventing bacterial infections in a mammal, which bacterial infections appear as co-infections with a virus, comprising contacting an affected area with a physiological saline solution, comprising (i) lysozyme, (ii) a pharmaceutically acceptable chelating agent, (iii) a pharmaceutically acceptable pH stabilizing salt, wherein the pH stabilizing salt has buffering capacity in the range of pH 3.0 to pH 7.0.

In one embodiment of the invention, the compounds of the present invention can be used as a therapeutic agent for bacterial infections that accompany any viral infection. In one preferred embodiment, the virus is one that causes a respiratory infection. Examples of viruses that cause respiratory infections include, but are not limited to influenza, paramyxovirus (e.g., respiratory syncytial virus, parainfluenza, metapneumovirus, picornavirus (enterovirus, rhinovirus), coronavirus (229E, NL63, OC43, HKU1, MERS-CoV, SARS-CoV, SARS-CoV-2 [causing COVID-19 syndrome], adenovirus and parvovirus.

If the compositions of the present invention are administered right at the beginning of the viral infection, the complex cooperation between the viruses and the bacteria that may challenge the immune system (which increases the morbidity and mortality of the viral infection) will be avoided or minimized. Furthermore, continued administration of the composition periodically (e.g., once every 48 hours) after the initial shock treatment (e.g., three times, once every 8 hours) for the duration of the plateau of the viral infection, the viral-bacterial coinfection is avoided; which gives the immune system a chance to fight and win the battle against the virus.

If compositions of the present invention are administered with shock treatment (e.g., three times, once every 8 hours) after the onset, or at a critical period of the viral infection (when the viral-bacterial coinfection is already mounted), the synergistic action of the viruses and the bacteria will be halted, and the storm reaction of the immune system and further systemic and micro-anatomic damages avoided; diminishing the morbidity, and facilitating the recuperation of the patient.

The compounds of the present invention can be used as a therapeutic agent for all bacterial infections, whether or not they accompany a viral infection. However, in one embodiment, the compounds of the present invention can be used as a therapeutic agent for bacterial infections that do accompany a viral infection, as a means to diminish the acuteness and mortality of the disease by avoiding, hampering or stopping the synergistic cooperation of the viruses and the bacteria that overwhelm the immune response.

In yet more detail, the present invention is described by the following items which represent preferred embodiments thereof:

1. A pharmaceutical formulation for administration to a mammal, comprising:
about 2% by weight to about 80% by weight of lysozyme;
about 2% by weight to about 40% by weight of a pharmaceutically acceptable chelating agent;

about 0.7% by weight to about 20% by weight of a pharmaceutically acceptable pH stabilizing salt, wherein the pH stabilizing salt has buffering capacity in the range of pH 3.0 to pH 7.0; and wherein said weight percent is relative to the total weight of the pharmaceutical formulation.

2. The pharmaceutical formulation of item 1, wherein the formulation is dissolved in a pharmaceutically acceptable aqueous solution prior to administration to a mammal.

3. The pharmaceutical formulation of item 1, wherein the chelating agent is selected from the group consisting of ethylene diamine tetra-acetic acid (EDTA) salt, citrate salt, alginate salt, and a combination thereof.

4. The pharmaceutical formulation of item 1, wherein the pH stabilizing salt is selected from the group consisting of citrate salt and sodium bicarbonate.

5. The pharmaceutical formulation of item 1, wherein the formulation further comprises a resin.

6. The pharmaceutical formulation of item 1, wherein the formulation further includes zinc oxide.

7. The pharmaceutical formulation of item 1, wherein the formulation further includes magnesium citrate.

8. The pharmaceutical formulation of item 5, wherein the formulation comprises a chewable tablet form.

9. The pharmaceutical formulation of item 2, wherein the formulation is provided in a nebulizer.

10. The pharmaceutical formulation of item 5, wherein the formulation comprises a coloring agent.

11. The pharmaceutical formulation of item 5, wherein the formulation comprises a flavoring agent.

12. The pharmaceutical formulation of item 1, wherein lysozyme is present in an amount of 80% weight percent or less relative to the total weight of the pharmaceutical formulation.

13. A method of treating or preventing a bacterial infection in a mammal, comprising: administering to an infected area of the mammal a pharmaceutical composition comprising:
i. about 2% by weight to about 80% by weight of lysozyme,
ii. about 2% by weight to about 40% by weight of a pharmaceutically acceptable chelating agent,
iii. about 0.7% by weight to about 20% by weight of a pharmaceutically acceptable pH stabilizing salt, wherein the pH stabilizing salt has buffering capacity in the range of pH 3.0 to pH 7.0.

14. The method of Item 13, wherein the infection is a skin infection.

15. The method of Item 14, wherein the composition is in physiological saline solution.

16. The method of Item 13, wherein the composition is a pharmaceutical powder formulation.

17. The method of Item 16, wherein the composition further comprises zinc oxide.

18. The method of Item 17, wherein the skin is dried prior to administering the composition.

19. The method of Item 13, wherein the infection is a respiratory tract infection.

20. The method of Item 19, wherein the composition is administered with a nebulizer.

21. The method of Item 13, wherein the composition is dissolved in water.

22. The method of Item 21, wherein the infection is a digestive tract infection.

23. The method of Item 22, wherein the composition further comprises magnesium citrate.

24. The method of Item 21, wherein the infection is in the large intestine.

25. The method of Item 21, wherein the infection is in the oropharyngeal mucosa.

26. The method of Item 13, wherein the infection is in the oral cavity.

27. The method of Item 26, wherein the composition is administered orally.

28. The method of Item 27, wherein the composition is in the form of a chewable pharmaceutical tablet.

29. The method of Item 28, wherein the composition further comprises a pharmaceutically acceptable resin.

30. The method of item 15, wherein the saline solution comprises about 100 mg to about 300 mg of the composition per liter of solution.

31. The method of item 15, wherein the saline solution is introduced into the bloodstream.

32. The method of item 31, wherein said treatment is for septicemia.

33. The method of item 15, wherein the infection is in one or more vital organs.

34. The method of item 15, wherein the solution is introduced into a surgical site during a surgical procedure.

35. The method of item 21, wherein the infection is in the eye.

36. The method of item 35, wherein the infection is conjunctivitis.

37. The method of item 13, wherein the bacterial infection accompanies a viral infection.

38. The method of item 37, wherein the viral infection comprises a respiratory tract infection.

39. The method of item 38, wherein a virus causing the viral infection is influenza, coronavirus, adenovirus and parvovirus.

40. The method of item 39, wherein the virus is a coronavirus.

41. The method of item 40, wherein the virus is MERS-CoV, SARS-CoV, or SARS-CoV-2.

42. The method of item 41, wherein the virus is SARS-CoV-2.

43. The method of item 42, wherein the virus causes COVID-19.

The compositions and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the processes, formulations and/or methods of the invention may be made without departing from the spirit of the invention and the scope of the appended claims.

### EXAMPLES

### Example 1

An example was prepared to compare the efficacy of a mouth rinse according to the formulation of the present invention (“Lysodent™”) and a Comparative Example (Digluconate 0.12%, “Chlorhexidine”) in Dental Surgery and Periodontal Disease. Lysodent™ was used alone in each study. Chlorhexidine was used as mouthwash during surgery, and Chlorhexidine Plus (Chlorhexidine+analgesic+antibiotic+anti-inflammatory) during the post-chirurgical recovery follow-up.

Preparation of Lysodent™ is shown in Table 1.

TABLE 1

| Component | Formulation (mg) |
|---|---|
| lysozyme | 36.0 |
| EDTA tetrasodium salt | 12.0 |
| Sodium Bicarbonate | 2.0 |
| Flavoring agent | 0.0 |
| Coloring agent | 0.0 |
| Total weight | 50 mg |

Study Population

Seventy subjects (gingivitis, n=40; surgical, n=30) were invited to participate in this clinical trial. Subjects were selected in a manner that allowed comparability and impartial analysis. The Clinical Examiner divided the subjects into two groups, depending on diagnosis—Gingivitis or Surgical candidate. Table 2 shows the selection criteria for the study population.

TABLE 2

| Inclusion criteria: | Exclusion criteria: |
|---|---|
| Be between 18 and 90 years of age. | Patient pregnant or nursing |
| Present with a Gingival Index (GI) | Experiencing chronic or systemic |
| score of at least 0.5, determined | disease that may impact surgical |
| upon examination; or | treatment or healing, as determined |
| Be diagnosed with Periodontal | by the clinical examiner |
| Disease needing an implant or | Patient using antibiotics within two |
| extraction. | weeks of baseline, visit 1. |

Patient Recruitment and Informed Consent Process

This study was approved by the Research Ethics Committee (ETIKOS) on Jun. 21, 2018. Patients who currently attend the Department of Surgery and Periodontics at EIBEGO were invited to be part of the study. Subjects were given an informed consent form and the investigators were available to answer any questions they may have had. Only after the subject fully understood the benefits and risks of the study, they were asked to sign the informed consent form, and a copy was given to them for their records. Subjects were also notified of their right to withdraw from the study at any point if they wish, without any repercussion to future treatment at the EIBEGO clinic.

Baseline, Visit 1. After the Informed Consent Process, medical and dental history was obtained. Comprehensive oral examination were conducted which included the Loe-Silness gingival index, and x-rays. Subjects were then randomly divided into two treatment groups (receiving Lysodent™ or Chlorhexidine). Subjects who received Lysodent™ were instructed to make a mouth rinse by combining 50 mg of Lysodent™ (pre-packaged in envelopes) with water using the pre-measured dose cup provided in their product kit. Subjects were asked to rinse for one minute, twice a day for 28 days. The first rinse in the morning, after breakfast and brushing their teeth and the second rinse at night, before bed. Subjects who received Chlorhexidine were instructed to use 15 ml of the mouth rinse three times a day for one minute, using the pre-measured dose cup provided in their product kit. They were asked to rinse in the morning before breakfast and brushing their teeth, 8 hours later, and before bed.

Visit 2—Three days after product use. Product usage compliance was assessed, and subjects were reinstructed, if needed. Subjects were evaluated using a comprehensive oral exam including the Loe-Silness gingival index. A survey was then conducted to record pain levels (if any), flavor comments and overall product perception. Subjects were asked to continue product use at home as instructed.

Visit 3—Seven days after surgery and product use. Surgical site was then photographed, and a survey was then conducted to record pain levels (if any), flavor comments and overall product perception. Subjects were instructed to discontinue product use after this visit.

Visit 4—Fourteen days after surgery and product use. Surgical site was then photographed, and a survey was then conducted to record pain levels (if any), flavor comments and overall product perception. This was the final visit and subjects were dismissed from the study.

Study Methods

Loe-Silness Gingival Index

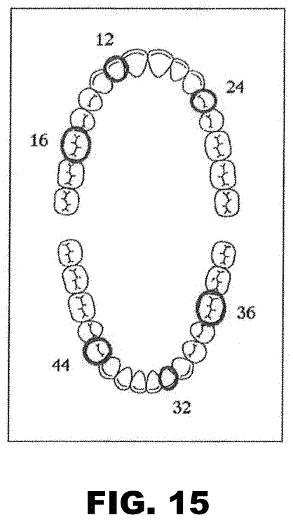

The measurement of the state of oral hygiene by Loe-Silness plaque index is based on recording both soft debris and mineralized deposits on the teeth shown in FIG. 15. Missing teeth are not substituted. Each of the four surfaces of the teeth (buccal, lingual, mesial and distal) are given a score from 0-3. The scores from the four are added and divided by four in order to give the plaque index for the tooth with the following scores and criteria shown in Table 3.

TABLE 3

| Score | Criteria |
|---|---|
| 0 | No plaque |
| 1 | A film of plaque adhering to the free gingival margin and |
| | adjacent area of the tooth. The plaque may be seen in situ |
| | only after application of disclosing solution or by using |
| | the probe on the tooth surface. |
| 2 | Moderate accumulation of soft deposits within the |
| | gingival pocket, or the tooth and gingival margin |
| | which can be seen with the naked eye. |
| 3 | Abundance of soft matter within the gingival pocket and/ |
| | or on the tooth and gingival margin. |

Study Results

Gingivitis Group

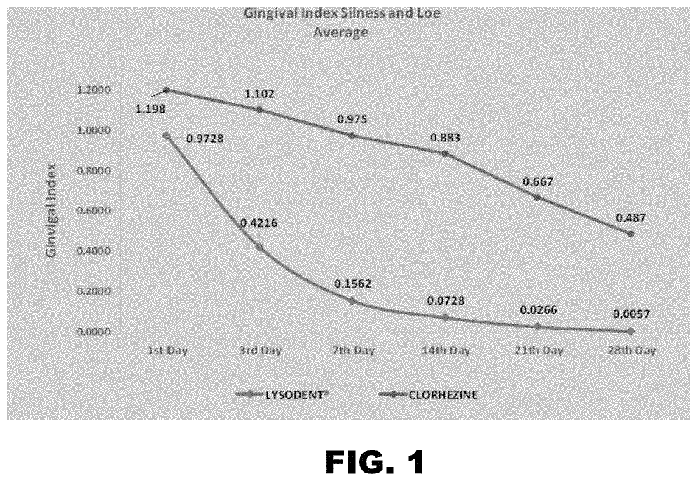

Comparison of patients with Gingivitis that underwent treatment with mouthwash using either Lysodent™ or Chlorhexidine, over 14 days, during and after undergoing dental clinical prophylaxis. Lysodent™ was much more effective in the treatment of gingivitis. At visit 2 (3 days of Lysodent™ treatment) subjects had a GI (Loe-Silness Index) score below 0.5, which categorized them as healthy patients in terms of periodontal disease (Gingivitis) (FIG. 1). At visit 4 (fourteen days of Lysodent™ treatment), no bleeding sites were reported, while gingival inflammation was minimized in 75% of all cases. At the end of the study, all subjects had a GI score either zero or approaching zero.

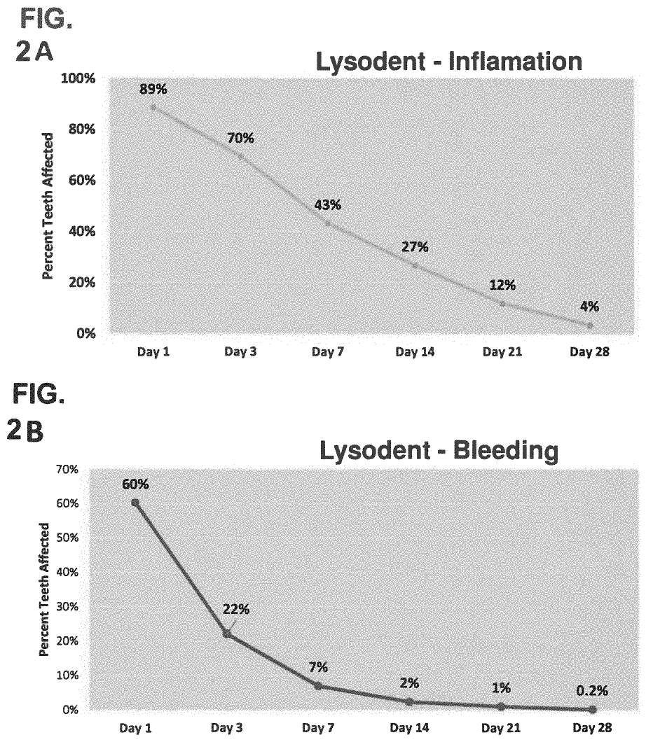

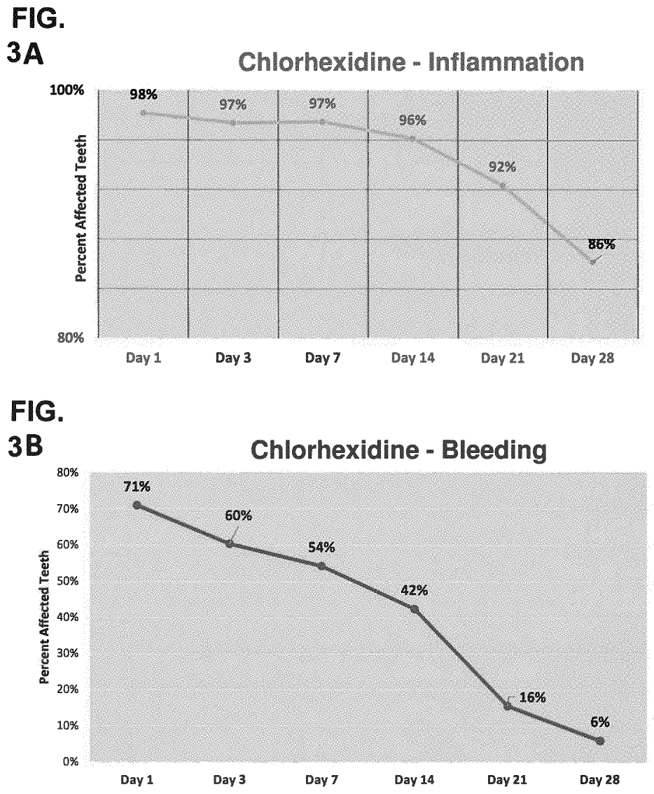

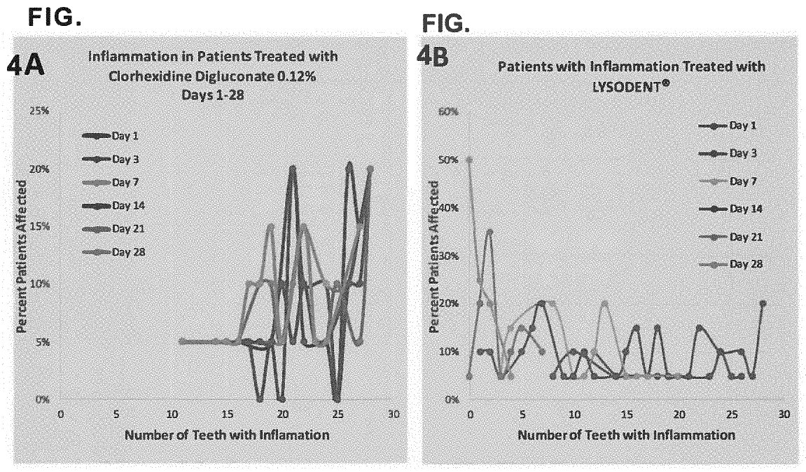

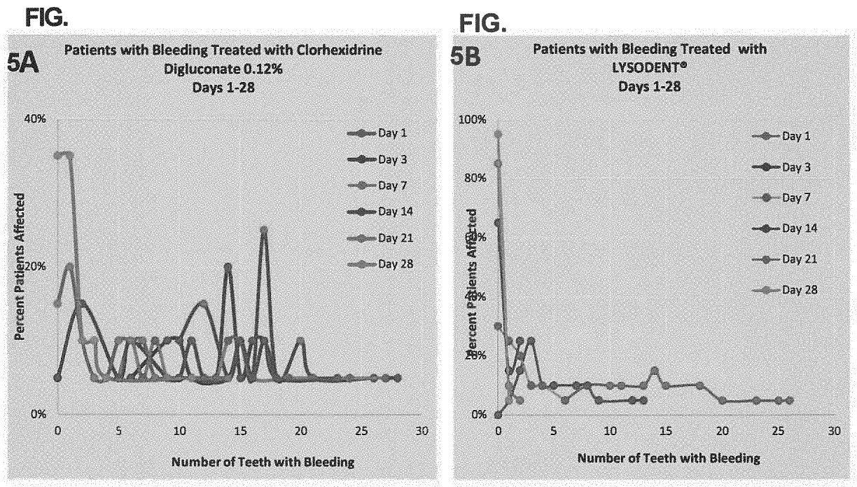

In contrast to Lysodent™ (FIGS. 2A and 2B), Chlorhexidine treatment showed a very slow recovery in terms of eliminating gingival inflammation and bleeding (FIGS. 3A and 3B). At the end of the study (28 days of treatment), almost 50% of the Chlorhexidine-treated subjects still had a GI score higher than 0.5 (FIG. 1). Patients treated with Chlorhexidine had an increased number of teeth with bleeding and inflammation throughout the 28 day study (FIGS. 4A and 5A) compared to patients treated with Lysodent™ (FIGS. 4B and 5B).

Dental Extraction Surgical Group

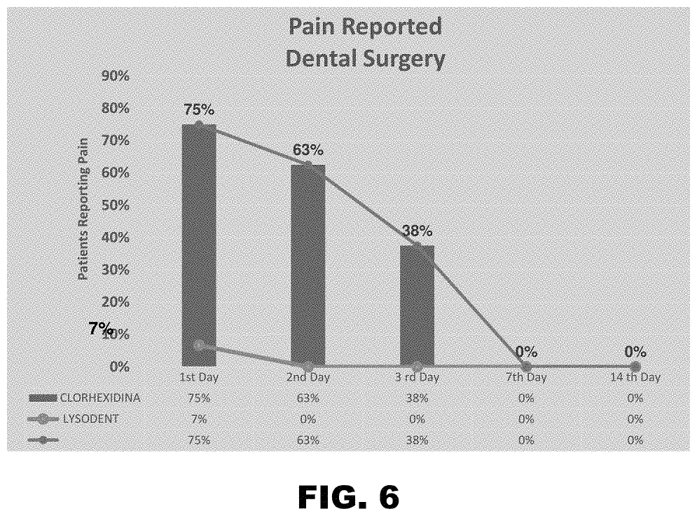

Lysodent™ was more effective in controlling pain associated with dental surgery compared to Chlorhexidine Plus. Only one of the Lysodent™ treated cases (7%) reported mild pain on the first day after initiating the use of the medication, as opposed to the number of reports in the cases under Chlorhexidine Plus (75% the first day after the medication started, 63% on the second day and 38% the third) (FIG. 6).

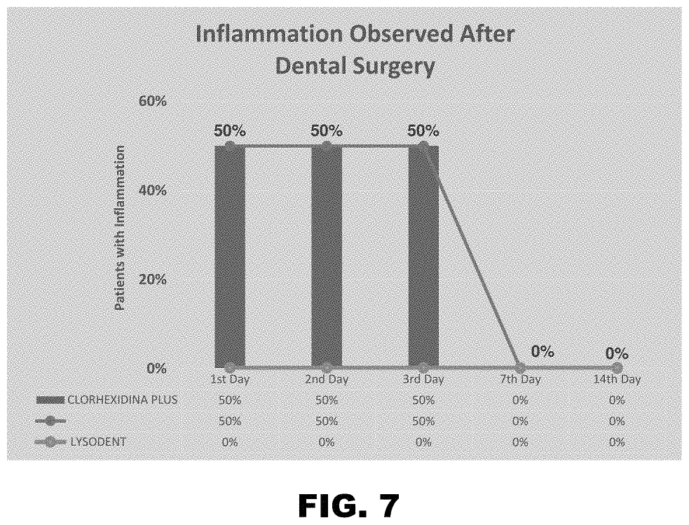

Lysodent™ proved to be superior for the prevention of inflammation (FIG. 7), since no cases of inflammation were reported following the start of the treatment, while 50% of the patients using Chlorhexidine Plus reported inflammation in the first three days.

Effectiveness for the prevention of bleeding could not be evaluated in the study. No patients reported bleeding during the study.

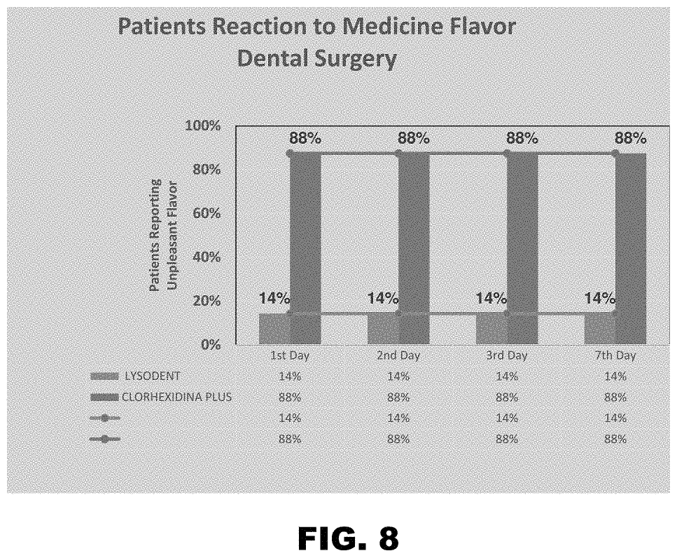

Lysodent™ proved to be better in the patient's assessment of the product flavor. Only 14% of the patients using Lysodent™ reported unpleasant taste of the mouthwash, while 88% of those using Chlorhexidine Plus reported unpleasant flavor (FIG. 8).

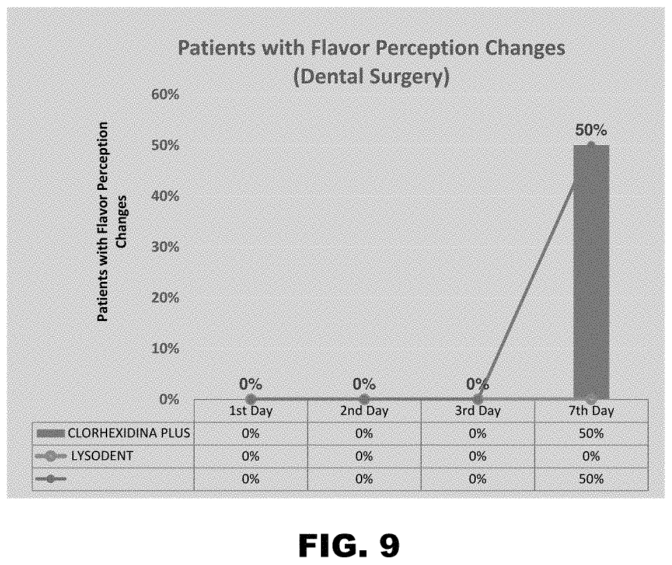

Lysodent™ did not affect the perception of flavor in any of the cases, while 50% of the cases using Chlorhexidine Plus reported alteration in the perception of food flavor (bitter mouth) after the seventh day of use (FIG. 9).

Dental Implant Surgical Group

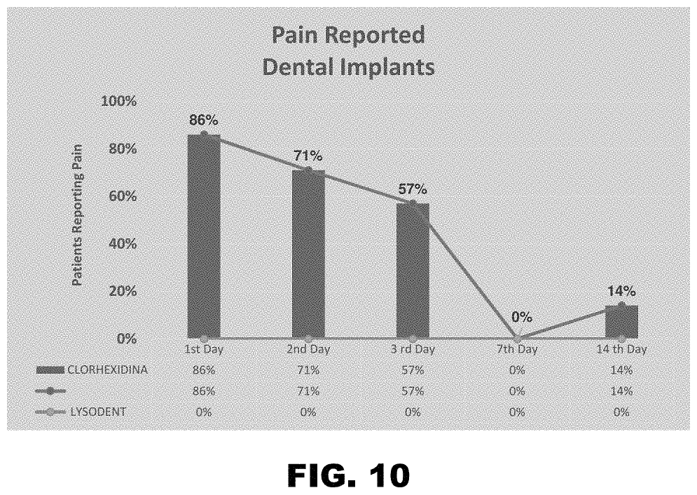

Lysodent™ proved to be much more effective in controlling pain than the combination of Chlorhexidine Plus. None of the Lysodent™ treated cases (0%) reported pain after initiating the use of the medication, as opposed to Chlorhexidine Plus, which included 86%, the first day after the medication started, 71% on the second day, 57% the third day, and 14% on the fourteenth day (FIG. 10).

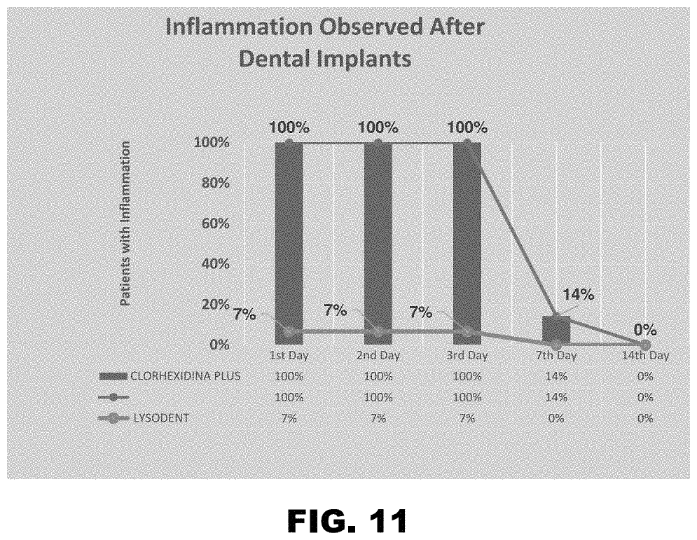

Lysodent™ proved to be superior for the prevention of inflammation, since only one case (7%) reported mild inflammation the first day through the third day following the start of the treatment, while 100% of the users of Chlorhexidine Plus reported inflammation in the same period (FIG. 11).

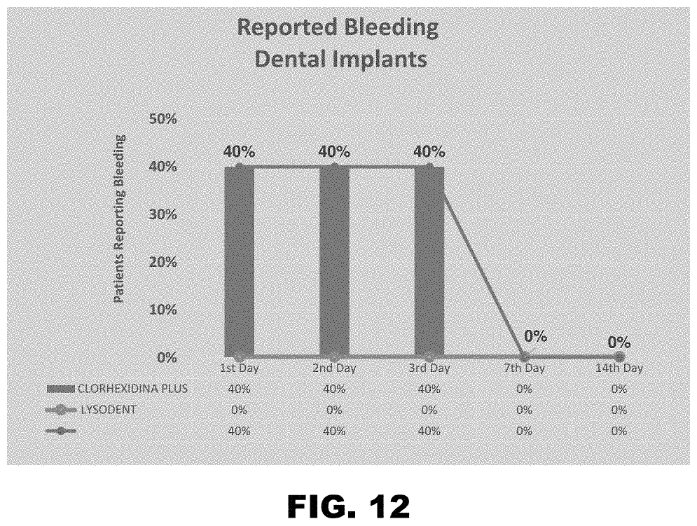

Lysodent™ also proved to be superior in the prevention of bleeding, since no patient reported bleeding, while 40% of the patients using Chlorhexidine Plus reported bleeding during the first three days of the study (FIG. 12).

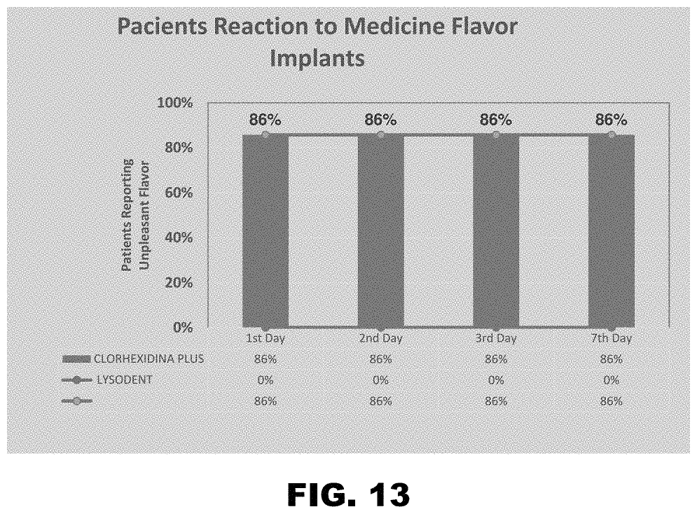

Patient assessment of the flavor of the product was also better for Lysodent™. None of the patients using Lysodent™ reported unpleasant taste of the mouthwash, while 86% of those using Chlorhexidine Plus reported unpleasant flavor (FIG. 13).

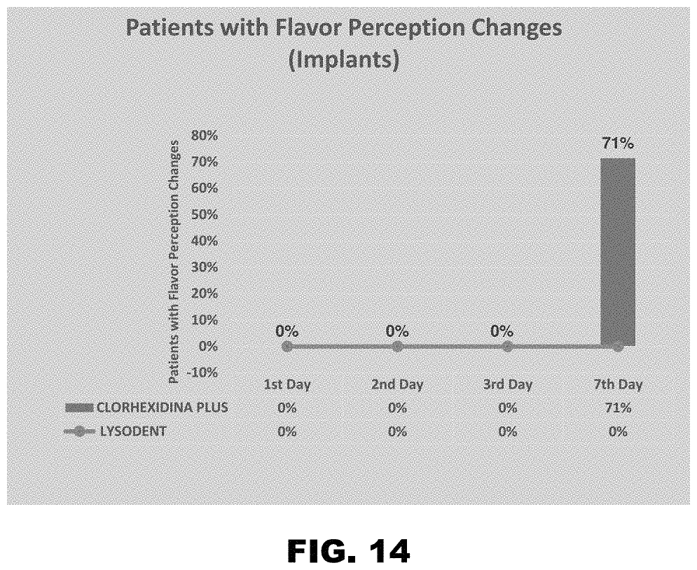

Moreover, Lysodent™ did not affect the perception of flavor in any of the cases, while 71% of the cases using Chlorhexidine Plus reported alteration in the perception of food flavor after the seventh day of use (FIG. 14).

The results of the studies show the effectiveness of Lysodent™ for eliminating the presence of bacteria in the oral cavity during surgery and during post-surgery recovery. It was found that treatment using Lysodent™ prevented symptoms associated with infection or high bacterial population in the oral cavity during and after surgery (pain, inflammation and bleeding). All the results point at the conclusion that Lysodent™ is safe and highly effective when used as an asepsis agent during surgery, and during the post-surgery follow-up to avoid infection of the surgical area.

In addition, the results of the study described herein shows that using Lysodent™ as an asepsis agent during and after surgery provides a rapid and efficient recovery and healing of the oral tissues. This effect is due to the almost complete asepsis produced by irrigating during surgery and the use of mouth rinse during recovery.

It is also notable that no adverse events were reported due to the use of Lysodent™ None of its components were found to be harmful to human tissue nor the metabolism of the human body. As a result, the present study provides evidence that Lysodent™ and formulations of the present disclosure can be swallowed after using as a mouth rinse. It is important to note that due to its effectiveness in eliminating the sources of infection, the need for post-surgical analgesics and antibiotics is unnecessary.

### Example 2

Application to the Sinus and Lungs Acute or Chronic Bacterial Infections.

LYSIBIOTIC is one composition of the present invention. Although LYSIBIOTIC is not harmful to the mucous surface of the lungs and it can be applied to any bacterial lung infection, this application is important when the bacteria strains involved are antibiotic resistant or when the patient is sensitive to antibiotics. LYSIBIOTIC has been applied to patients with chronic and very acute lung infections with full recovery in less than 48 hours; furthermore, LYSIBIOTIC is very useful in eliminating the bacterial coinfection of viral respiratory infections. Because it does not produce any kind of side effect, LYSIBIOTIC is the best choice when the patient is immunodeficient or is undergoing chemotherapy.

Usage Procedure.

The most effective way for delivering the LYSIBIOTIC formula to the lungs is through a Nebulizer. Dissolving one dose (100 mg) of LYSIBIOTIC in distilled water or physiological saline solution has proven highly effective (in 100% of the cases full recovery has been achieved). The application procedure is as follows: Dissolve 100 mg of the product in 5 ml distilled water or physiologic saline solution and apply with a nebulizer; repeat the application every eight hours until it has been applied three times. In most cases it is not necessary to apply the formula more than three times.

### Steps for Implementation of the Protocol

Step I: Eligibility Criteria: The persons eligible to enter the study are patients who meet the following characteristics:
1. Patients over the age of 15 of both sexes and any race or religion and who entered a hospital with a diagnosis of COVID-19 caused by SARS-CoV-2 and based on the criteria cited below.
2. Positive COVID-19 test (Any fast/quick tests or RT-PCR).
3. People with acute respiratory disease with sudden onset of at least one of the following symptoms:
   a. Cough
   b. Sore throat,
   c. Difficulty breathing or
   d. Fever (Subjective) o≥38° C.
4. Patient is admitted with severe pneumonia of unknown origin and/or evidence of imaging injury compatible with pneumonic infiltrate (new and/or persistent alveolar infiltration) and/or signs and symptoms of pulmonary consolidation.
5. Written and signed consent by the patient or family member responsible for the use of Lysibiotic; inform of the characteristics of the study (complying with all ethical guidelines, including those of the Helsinki Protocol), voluntarily agreeing to participate in the same.

Exclusion Criteria:
1. Patients who request their exclusion from the use of the drug.
2. Patients who, because of their condition, the doctor deems it appropriate to suspend their participation in the study (mainly because of the form of administration of the drug Lysibiotic).
3. Hypersensitivity to the medicinal product (not reported).

Step II: Written consent of the Patient or Responsible Family: The patient or family member responsible is thoroughly informed of all aspects involved in the Lysibiotic Procedure (including those of the Helsinki Protocol). The procedure is tested for effectiveness in treating pneumonia in general, as well as COVID-19 pneumonia. The authorized patient or family member is asked to read and sign the consent document if He (she) believes he or she agrees to submit the patient to the Lysibiotic Procedure.

Step III: The study is conducted with two groups: Group A, standard group for comparison. This group follows the standard treatment that the hospital uses for these patients. Group B, group under study, this group follows standard treatment minus antibiotics (standard hospital treatment eliminating antibiotic use), plus Lysibiotic Treatment.

Step IV: Bioanalytics and Imaging: Before starting the procedure, the patient is evaluated with bioanalytical and imaging tests to determine the following parameters:
1) Complete blood count
2) Glycemia
3) Erythrocyte Sedimentation rate (ESR)
4) Hemoglobin (HbA1c)
5) Renal profile:
   a. Urinalysis
   b. Urea
   c. Creatinine
   d. Uric Acid
6) Hepatic profile:
   a. Alanine Aminotransferase (ALT)—formerly called serum glutamic-pyruvic transaminase (SGPTb)-
   b. Aspartate Aminotransferase (AST)—formerly called serum glutamic-oxaloacetic transaminase (SGOTc)-
   c. Bilirubin
7) Thyroid profile
   a. Triiodothyronine (T3)
   b. Thyroxine (T4 Free)
   c. Thyroid Stimulating Hormone (TSH)
   d. Thyroglobulin 8) Lipid Profile:
a. Cholesterol,
b. Triglycerides
c. High-density lipoprotein (cholesterol HDL)
d. Low-density lipoprotein (cholesterol LDL)
e. Very low-density lipoprotein (cholesterol VLDL)
9) Polymerase chain reaction (PCR)
10) Procalcitonin (PCT)
11) Electrolytes
12) Creatine phosphokinase (CPK)
13) Coagulation Profile
14) Ferritin
15) Dimer D
16) Enzyme-Linked Immunosorbent Assay (ELISA) for human immunodeficiency viruses (HIV)
17) Blood cultures
18) Nasal swabbing for Staphylococcus Aureus methicillin-resistant (SAMR)
19) Electrocardiogram (EKG)
20) Diagnostic hysterolaparoscopy (DHL)
21) Thorax Posteroanterior (PA) Radiography or Thorax Computed axial tomography (CAT)/Computed tomography (CT) TAC (HD) Thorax
22) Arterial Gases

Step V: The Treatment (Group B) is applied as follows:
a. 100 mg (one hundred milligrams) of Lysibiotic 100 ml in powder is dissolved in 5 ml (five milliliters) of physiological saline solution, shaken gently until it dissolves completely and placed in the nebulizer container.
b. Place the patient in the nebulizer and turn it on. Make sure the patient stays nebulized until the container is empty.
c. 8 (eight) hours after first Nebulization, repeat Nebulizations steps a) and b).
d. 8 (eight) hours after second Nebulization, repeat Nebulizations steps a) and b).
e. Wait 8 (eight) hours and repeat steps IV (Bioanalytics and Imaging).
f. 48 hours after last Nebulization, repeat Nebulizations steps a) and b).
g. Wait 8 (eight) hours and repeat steps IV (Bioanalytics and Imaging).
h. 48 hours after last Nebulization, repeat Nebulizations steps a) and b).
i. Wait 8 (eight) hours and repeat steps IV (Bioanalytics and Imaging).
j. 48 hours after last Nebulization, repeat Nebulizations steps a) and b).
k. Wait 8 (eight) hours and repeat steps IV (Bioanalytics and Imaging).

From step k), depending on the indicators results of the k step, patients are followed and treated based on the hospital's COVID-19 management protocol.

Nebulizations in COVID-19 patients are done under strict spray control, in a room with negative pressure and ideally with Capacete or Hood Cephalic Chamber for adults and complete protection from the executing physician, preferably with FFP3 mask: provide a maximum filter efficiency of around 98%, and a maximum total leakage rate of 2%; or N-95.

Group B, apart from following the Lysibiotic Treatment, continue the same treatment as Group A, except that it will be eliminated from the supply of antibiotics.

Group A follows the hospital's standard treatment for COVID-19 patients.

During the study, clinical, bioanalytical and imaging data from both Group A and Group B patients are compared according to daily developments. All parameters of clinical evaluations resulting from steps e), g), (i) and k) of V, are analyzed and compared to those in step IV.

The patent and scientific literature referred to herein establishes the knowledge that is available to those with skill in the art. Any United States patents and published or unpublished United States patent applications cited herein are incorporated by reference. Any published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A pharmaceutical formulation for administration to a mammal, comprising:
about 2% by weight to about 80% by weight of lysozyme;
about 2% by weight to about 40% by weight of a pharmaceutically acceptable chelating agent;
about 0.7% by weight to about 20% by weight of a pharmaceutically acceptable pH stabilizing salt, wherein the pH stabilizing salt has buffering capacity in the range of pH 3.0 to pH 7.0; and wherein said weight percent is relative to the total weight of the pharmaceutical formulation.

2. The pharmaceutical formulation of claim 1, wherein the formulation is dissolved in a pharmaceutically acceptable aqueous solution prior to administration to a mammal.

3. The pharmaceutical formulation of claim 1, wherein the chelating agent is selected from the group consisting of ethylene diamine tetra-acetic acid (EDTA) salt, citrate salt, alginate salt, and a combination thereof.

4. The pharmaceutical formulation of claim 1, wherein the pH stabilizing salt is selected from the group consisting of citrate salt and sodium bicarbonate.

5. The pharmaceutical formulation of claim 1, wherein the formulation further comprises a resin.

6. The pharmaceutical formulation of claim 1, wherein the formulation further includes zinc oxide.

7. The pharmaceutical formulation of claim 1, wherein the formulation further includes magnesium citrate.

8. The pharmaceutical formulation of claim 1, wherein the formulation comprises a chewable tablet form.

9. The pharmaceutical formulation of claim 1, wherein the formulation is provided in a nebulizer.

10. The pharmaceutical formulation of claim 1, wherein lysozyme is present in an amount of 80% weight percent or less relative to the total weight of the pharmaceutical formulation.

11. A method of treating or preventing a bacterial infection in a mammal, comprising:
administering to an infected area of the mammal a pharmaceutical composition comprising:
i. about 2% by weight to about 80% by weight of lysozyme,
ii. about 2% by weight to about 40% by weight of a pharmaceutically acceptable chelating agent,
iii. about 0.7% by weight to about 20% by weight of a pharmaceutically acceptable pH stabilizing salt, wherein the pH stabilizing salt has buffering capacity in the range of pH 3.0 to pH 7.0.

12. The method of claim 11, wherein the infection is a respiratory tract infection.

13. The method of claim 11, wherein the composition is administered with a nebulizer.

14. The method of claim 11, wherein the composition is administered into the bloodstream.

15. The method of claim 11, wherein the composition is introduced into a surgical site during a surgical procedure.

16. The method of claim 11, wherein the administering treats or prevents septicemia.

17. The method of claim 11, wherein the bacterial infection accompanies a viral infection.

18. The method of claim 17, wherein a virus causing the viral infection is influenza, coronavirus, adenovirus and parvovirus.

19. The method of claim 18, wherein the virus is SARS-CoV-2.

20. The method of claim 19, wherein the virus causes COVID-19.
